(19) United States
(12) Patent Application Publication
(10) Pub. No.: US 2019/0174824 A1
(43) Pub. Date: Jun. 13, 2019

(54) Botanic Solid Vapor Generation Apparatus

(71) Applicants: Bradley Aaron Neuhaus, North Hollywood, CA (US); Marshal Glenn Heingartner, North Hollywood, CA (US)

(72) Inventors: Bradley Aaron Neuhaus, North Hollywood, CA (US); Marshal Glenn Heingartner, North Hollywood, CA (US)

(21) Appl. No.: 15/839,686

(22) Filed: Dec. 12, 2017

(51) Int. Cl. A24F 47/00; A24F 1/30; A61M 15/06

(52) U.S. Cl. CPC A24F 47/008; A24F 7/02; A61M 15/06; A24F 1/30

(57) ABSTRACT

A vapor generator for generating vapors from a solid fuel is described. The vapor generator may comprise a fuel chamber for containing the fuel, an electrical heating element, associated controls, and a battery. The fuel is heated by ambient air drawn past the heating element, and is subsequently conducted into the fuel chamber. A screen may interpose a barrier between the fuel and the heating element, so that fuel is heated only convectively by air and not by direct contact with the heating element. The fuel chamber is modularly detachable from the heating element. Modules including the heating element and the fuel chamber may be joined by a ball and socket arrangement, thereby enabling ready assembly into a unitized assembly, and disassembled to enable the heating elements and related components to be utilized with a pre-existing smoking pipe or cigarette.

### FIELD OF THE INVENTION

The present invention relates to apparatus for selectively generating a vapor or smoke from a botanic solid, and more particularly, to a modular apparatus limiting heat used to generate the vapor or smoke, and usable in as a self-contained or stand-alone device, or alternatively, with a conventional smoking pipe.

### BACKGROUND OF THE INVENTION

People enjoy generating smoke from botanic solids, and inhaling this smoke. Pipes and hookahs for smoking have been developed for this purpose. However, pipes and hookahs can combust the latter, thereby altering smoke characteristics and rapidly and unnecessarily depleting the botanic solid.

### SUMMARY OF THE INVENTION

The present invention contemplates improved apparatus for generating vapors and smoke from herbiferous solids. Notably, the apparatus can be utilized selectively with smoking pipes and also with its own separable or detachable vapor generating module. The apparatus includes a vapor generating module and a detachable heating module. The heating module may include an electrical heating element. The vapor generating module may include a fuel body including a fuel chamber for containing a solid fuel and a socket for connections. The heating source may be used with its specific detachable vapor generating module to vaporize fuel, or alternatively, with a conventional smoking pipe to both vaporize or combust fuel. The vapor generating module may comprise an interface member having capacity to store sufficient heat to vaporize the fuel. The interface member of the heating module may be configured substantially as a sphere that can be used to sealingly engage a traditional smoking pipe, or alternatively, to allow for the ball and socket engagement of the vapor generating module. The vapor generating apparatus may include a mouthpiece attachable to and detachable from the vapor generating module.

The present disclosure describes a vapor generator for generating vapors from a solid fuel under controllable temperature conditions, the vapor generator comprising: a fuel body including a fuel chamber for containing the fuel and subjecting the fuel to heat; a heating module comprising an enclosure, an electrical heating element, a battery, and an electrical circuit configured to conduct electrical power from the battery to the electrical heating element; and an airflow path enabling ambient air to be drawn first past the electrical heating element, then to the fuel chamber passage to generate a vapor from the fuel.

The present disclosure further describes a vapor generator for generating vapors from a solid fuel under controllable temperature conditions, the vapor generator comprising: a heating module comprising an enclosure, an electrical heating element, a battery, and an electrical circuit configured to conduct electrical power from the battery to the electrical heating element; a vapor generating module manually detachable from the heating module, the vapor generating module including a fuel chamber for containing the fuel and subjecting the fuel to heat; and a connector manually connecting the heating module to and manually disconnecting the heating module from the vapor generating module.

The present invention provides improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1A is a side environmental view of a vapor generator according to at least one aspect of the invention, illustrating use as a stand-alone device;

FIG. 1B is a side environmental view of a vapor generator according to at least one aspect of the invention, illustrating use in conjunction with a smoking pipe;

FIG. 2 is an exploded side cross sectional detail view of an element shown at the left of FIG. 1A;

FIG. 3 is a side cross sectional detail view similar to FIG. 2, but showing the element assembled;

FIG. 4 is a side view showing the vapor generator of FIG. 1 in an assembled condition, including a mouthpiece shown in FIG. 1A; and

FIG. 5 is an electrical schematic of a heating element of the vapor generator of FIG. 1, and associated support elements.

### DETAILED DESCRIPTION

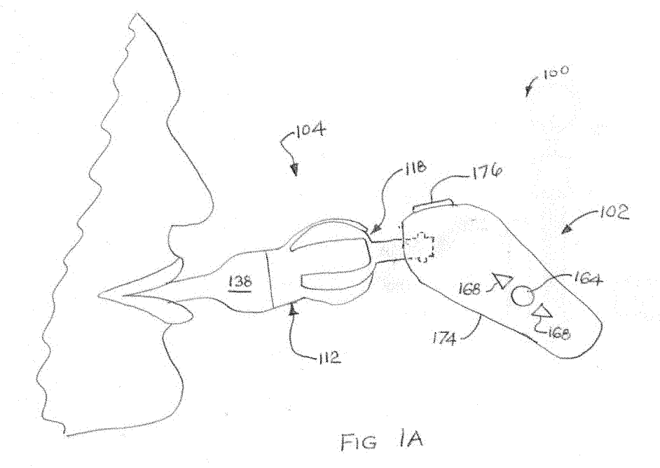

FIG. 1A shows a vapor generator 100 according to at least one aspect of the invention. Vapor generator 100 may include a heating module 102 for supplying heat to a botanic solid (not shown), and a vapor generating module 104 for holding and generating vapors from the botanic solid. Hereinafter, the botanic solid will be referred to as a fuel because the latter can combust, even though in many cases, vaporization is by pyrolysis with minimal actual combustion if any. The fuel may be tobacco, marijuana, or other botanic solids used for smoking. It will be seen from FIG. 1A that a vapor generating module 104 may be utilized with heating module 102 when it is desired to operate vapor generator as a self-contained unit. In FIG. 1B, optionally, heating module 102 is utilized with a conventional smoking pipe 106. Vapor generator 100 is considered to be in self-contained form when utilized as shown in FIG. 1A. In the self-contained form, vapor generator 100 is utilized with dedicated fuel holding apparatus (i.e., vapor generating module 104) not required when utilized with pipe 106, and preferably with a mouthpiece 138.

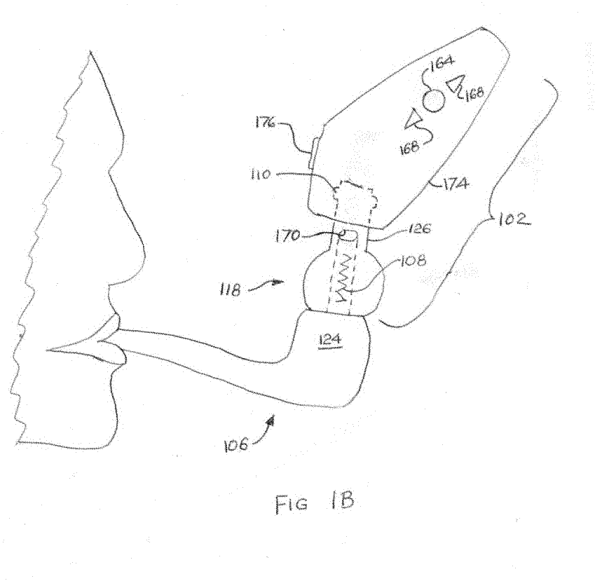

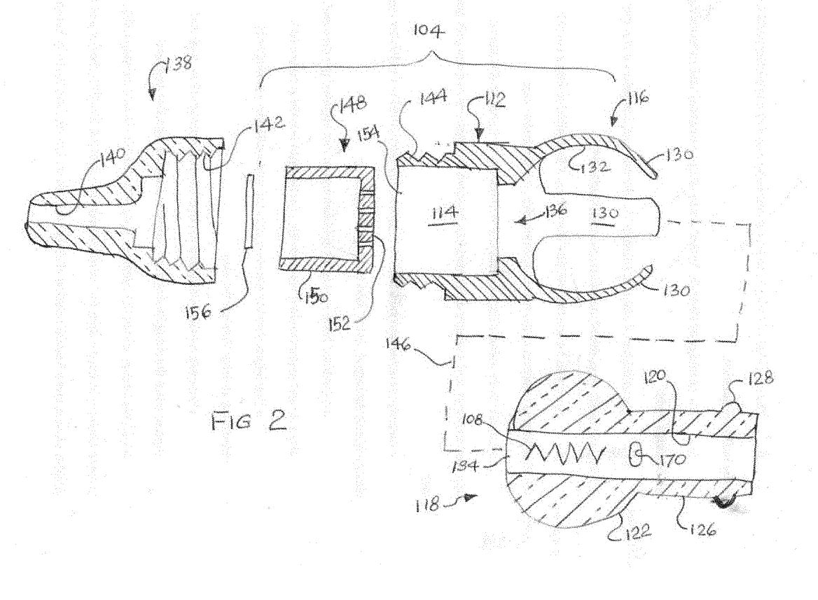

Referring also to FIGS. 2 and 3, vapor generator 100 may comprise heating module 102 including an electric heating element 108 (FIG. 1), an interface member 118 including a first internal passage 120 in fluid communication with electric heating element 108 and with ambient air at ambient air temperatures, and a tapered engagement surface 122 sealingly compatible with a bowl 124 of a smoking pipe 106.

Vapor generator 100 also comprises vapor generating module 104 manually attachable to and detachable from heating module 102. Vapor generating module 104 may include a fuel body 112 including a fuel chamber 114 for containing the solid fuel, a second internal passage 136 in fluid communication with fuel chamber 114 and with first internal passage 120 of interface member 118, and a socket 116 for engaging tapered engagement surface 122 of heating module 102. It must be borne in mind that interface member 118, although modularly removable from heating module 102, operates as part of the heating module 102, serving as a connector manually connecting heating module 102 to and manually disconnecting heating module 102 from vapor generating module 104.

As seen particularly in FIG. 2, interface member 118 includes a stem 126 bearing a flange 128. Stem 126 penetrates interface member 118 such that flange 128 engages a corresponding groove or connector 110 formed in an enclosure 174 of heating module 102 by resilient or snap fit. As seen in FIGS. 1A and 1B, electrical heating element 108 projects from heating module 102 and occupies internal passage 120 of interface member 118 when interface member 118 is assembled to heating module, and extends into the spherical portion of interface member 118.

Unless otherwise indicated, the terms “first”, “second”, etc., are used herein merely as labels, and are not intended to impose ordinal, positional, or hierarchical requirements on the items to which these terms refer. Moreover, reference to, e.g., a “second” item does not either require or preclude the existence of, e.g., a “first” or lower-numbered item, and/or, e.g., a “third” or higher-numbered item.

First internal passage 120 may be utilized to ignite a cigarette (not shown) or like device. The cigarette is inserted into first internal passage 120 after interface member 118 is heated sufficiently for ignition.

Ambient air passing into fuel chamber 114 is drawn through an opening 170 (FIGS. 1B, 2, 3, and 4) in stem 126. An airflow path enabling ambient air to be drawn first past electrical heating element 108, then to fuel chamber 114 to generate a vapor from the fuel is thus established. This is a significant feature of vapor generator 100, since it enables close control over temperatures acting on the fuel. Firstly, because heat may be applied in incremental steps, it is important to route air past heating element 108 prior to impinging on the fuel. Secondly, the user can draw more or less air into opening 170 and through interface member 118 and into fuel chamber 114. Varying volume of air flow will influence temperatures of that air.

Varying temperatures can result firstly, in selectively vaporizing or combusting the fuel. Also, it can be utilized to compensate for variations in fuel, such as moisture content, density with which fuel was packed into fuel chamber 114, type of fuel, and other factors. Hence it is significant to introduce air first past heating element 108 and subsequently into fuel chamber 114, and to separate heating element 108 from fuel chamber 114.

Socket 116 is defined within cooperatingly configured individual fingers 130 of fuel body 112. Socket 116 of vapor generating module 104 may comprise at least two fingers 130 resiliently cooperating with and capable of grasping and retaining interface member 118. Thus socket 116 can releasably close over interface member 118 of vapor generating module 102.

Three fingers 130 of five fingers provided in the illustrated embodiment are seen in FIG. 2, although any desired number of fingers 130 may be provided. Tapered engagement surface 122 corresponds to surrounding and close fitting surfaces of fingers 130, thereby establishing cooperation between socket 116 and fuel body 112.

In the embodiment illustrated in FIG. 2, an interface surface 132 of socket 116 of vapor generating module 104 comprises a generally spherical surface, thereby cooperating with generally spherical tapered engagement surface 122 of interface member 118. As employed herein, tapered engagement surface 122 is generally spherical in that an otherwise entirely spherical shape is necessarily altered by opening 134 (FIG. 2) and stem 126 of interface member 118. Alternatively stated, interface member 118 may have a generally spherical outer surface cooperating and engageable with a generally spherical surface of interface surface 132 (FIG. 2) of socket 116.

Spherical nature of socket 116 and interface member 118 provides one convenient way of securing vapor generating module 104 to heating module 102. Specifically, it enables a convenient snap fit which also holds vapor generating module 104 in a fixed orientation relative heating module 102. Therefore, detachable vapor generating module is readily attached and detached, and when attached, vapor generator 100 can be held by one hand by the user while smoking or vaping.

Interface surface 122 is tapered along an axis 146 (FIG. 2) of air flow. This provides a tapered surface formed in and facing away from heating module 102, whereby interface member 118 serves as a connector and also may be placed over and sealingly engage bowl 124 of pipe 106, as shown in FIG. 1B. This affords another mode of use, wherein a fuel is acted on by heat, but is not constrained to use only with vapor generating module 104.

In FIG. 1B, vapor generating module 102 encompasses interface member 118, the latter including an internal passage 120 in fluid communication with fuel chamber 114 and tapered engagement surface 122 sealingly compatible with bowl 124 of smoking pipe 106 and configured to cooperate with socket 116 of fuel body 112. Internal passage 120 is in fluid communication with ambient air and also with electric heating element 108 of heating module 102 when vapor generating module 104 is attached to heating module 102.

The connector provided by a partially spherical member may be in either one of heating module 102 and vapor generating module 104, as long as there is provided a socket 116 dimensioned and configured to receive and retain the partially spherical member in the other one of heating module 102 and vapor generating module 104. However, location of the partially spherical member as part of heating module 102 enables the use of the latter as an igniter for pipe 106.

Referring particularly to FIG. 2, vapor generator 100 may further comprise mouthpiece 138 attachable to and detachable from vapor generating module 104. Mouthpiece 138 may have a third internal passage 140 in fluid communication with fuel chamber 114 when vapor generating module 104 is assembled as seen in FIG. 3, and therefore, with second internal passage 136. Mouthpiece 138 may include threads 142. Vapor generating module 104 may include threads 144 compatible with threads 142 of mouthpiece 138. Thereby, mouthpiece 138 is removably attached to and detached from vapor generating module 104 by a threaded connection between mouthpiece 138 and fuel body 112.

Interface member 118 of vapor generating module 104 may be fabricated from a material impervious to temperatures high enough to induce vapor formation from fuel contained within fuel chamber 114. For example, interface member 118 of vapor generating module 104 may be fabricated from a ceramic material.

Vapor generator 100 may further comprise a fuel holder 148 (FIG. 2) dimensioned and configured to be received within fuel chamber 114. Fuel holder 148 is separate and removable from fuel body 112. Fuel holder 148 may comprise a lateral wall 150, a perforate end wall 152, and an open end 154 opposite perforate end wall 152. Perforate end wall 152 enables fuel to be exposed to heat by air heated by electrical heating element 108, and prevented direct contact of the fuel with heating element 108.

Vapor generator 100 may further comprise a screen 156 (FIG. 2) closing open end 154 of fuel holder 148. Screen 156 may be separate and removable from fuel holder 148. Screen 156 passes air and vapors from fuel chamber 114 to mouthpiece 138, but retains fuel within fuel holder 148.

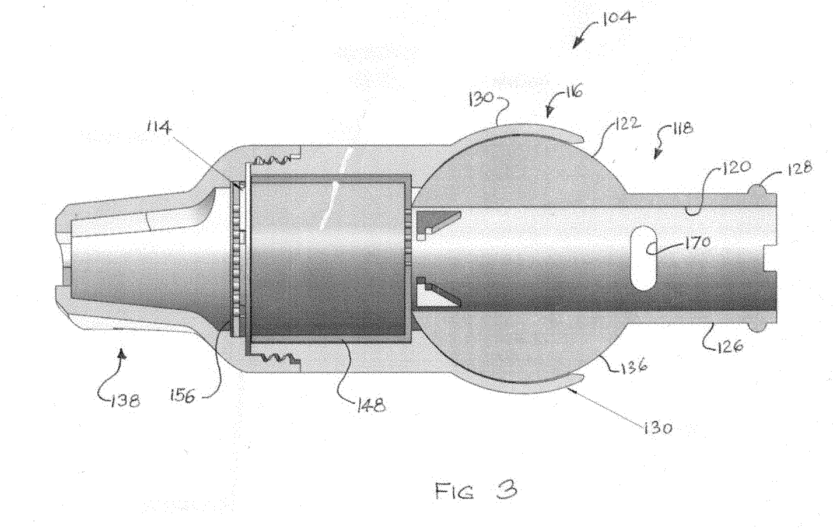

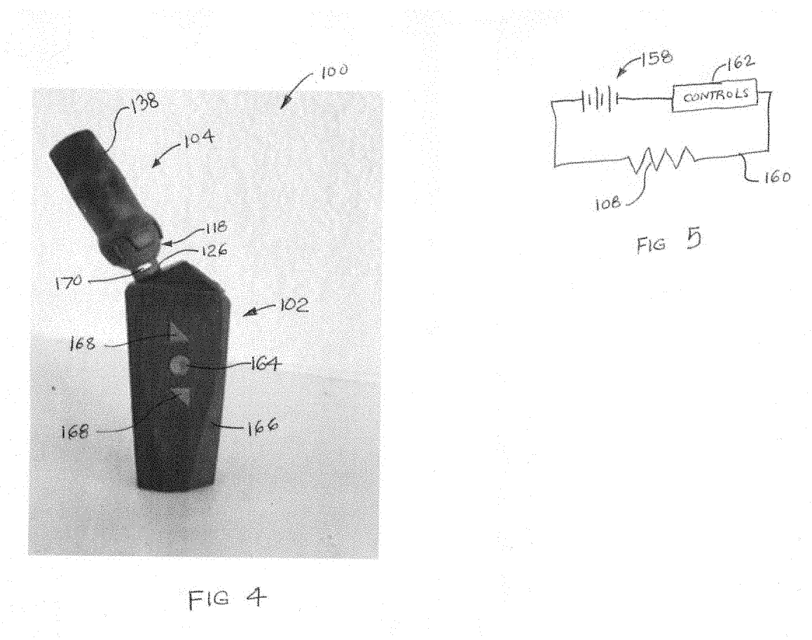

Referring also now to FIG. 5, heating module 102 (FIG. 4) may include a battery 158 and an electrical circuit 160 controlling current between battery 158 and electric heating element 108. Electrical circuit 160 may include controls 162 shown generically in FIG. 5. More specifically, electrical circuit 160 may include a first on-off control 164 (FIG. 4) accessible from an exterior 166 of heating module 102. Electrical circuit 160 may include manual controls 168 enabling increase and decrease in electrical power supplied to the heating element. Power may be increased and decreased in discrete steps (e.g., an additional increment of heat is applied or removed by repeated pressing of one of the manual controls 168). These steps may be annunciated by LED indicator lights (not shown) on enclosure 174 of heating module 102. A second on-off control 176 (FIGS. 1A and 1B) may be provided. Redundancy of first and second on-off controls 164 and 176 helps prevent unintended operation of heating element 108.

Thus there has been presented a versatile vapor generator 100 usable in three ways. First, vapor generator 100 may be utilized as a smoking or vaping pipe in its self-contained form. Secondly, heating module 102 may be utilized with a separate smoking pipe 106, where the user prefers to use his or her own pipe. Finally, under full or high heat output, the heating module 102 of vapor generator 100 may be utilized to ignite a cigarette or the like.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A vapor generator for generating vapors from a solid fuel under controllable temperature conditions, the vapor generator comprising:
a fuel body including a fuel chamber for containing the fuel and subjecting the fuel to heat;
a heating module comprising an enclosure, an electrical heating element, a battery, and an electrical circuit configured to conduct electrical power from the battery to the electrical heating element; and
an airflow path enabling ambient air to be drawn first past the electrical heating element, then to the fuel chamber passage to generate a vapor from the fuel.

2. The vapor generator of claim 1, wherein the heating module includes an interface member enabling detachable connection of the fuel body to the heating module, the interface member having an internal passage in fluid communication with the fuel chamber and the heating module.

3. The vapor generator of claim 2, wherein one of the interface member and the fuel body comprises a partially spherical member, and the other of the interface member and the fuel body comprises a socket dimensioned and configured to receive and retain the partially spherical member.

4. The vapor generator of claim 2, wherein the interface member comprises an opening for admission of ambient air into the air flow path.

5. The vapor generator of claim 2, further comprising a barrier permeable to gases and vapors separating the fuel from the electrical heating element, whereby fuel is exposed to heat by air heated by the electrical heating element and is prevented from direct contact with the heating element.

6. The vapor generator of claim 5, further comprising a fuel holder dimensioned and configured to be received within the fuel chamber, and wherein the fuel holder is separate and removable from the fuel body, the fuel holder comprising a lateral wall and an open end, and the barrier comprises a perforate end wall of the fuel holder opposite the open end.

7. The vapor generator of claim 6, further comprising a screen closing the open end of the fuel chamber, wherein the screen is separate and removable from the fuel body.

8. The vapor generator of claim 2, wherein the interface member is fabricated from a material impervious to temperatures high enough to induce vapor formation from fuel contained within the fuel chamber.

9. The vapor generator of claim 8, wherein the interface member is fabricated from a ceramic material.

10. The vapor generator of claim 1, further comprising a mouthpiece attachable to and detachable from the fuel body, the mouthpiece having an internal passage in fluid communication with the fuel chamber.

11. The vapor generator of claim 10, wherein the mouthpiece includes threads and the vapor generating module includes threads compatible with the threads of the mouthpiece, wherein the mouthpiece is removably attached to and detached from the fuel body by a threaded connection.

12. The vapor generator of claim 1, wherein the electrical circuit includes a first on-off control accessible from an exterior of the heating module.

13. The vapor generator of claim 12, wherein the electrical circuit includes manual controls enabling increase and decrease in electrical power supplied to the heating element.

14. The vapor generator of claim 12, further comprising a second on-control accessible from the exterior of the heating module, wherein the second on-off control is spaced apart from the primary on-off control.
